(19) United States
(12) Patent Application Publication
(10) Pub. No.: US 2022/0289826 A1
(43) Pub. Date: Sep. 15, 2022

(54) CHIMERIC CYTOKINE RECEPTOR CAPABLE OF IMMUNE SIGNAL CONVERSION, IMMUNE CELLS EXPRESSING SAME, AND ANTI-CANCER USE THEREOF

(71) Applicants: CHUNGBUK NATIONAL UNIVERSITY INDUSTRY-ACADEMIC COOPERATION FOUNDATION, Cheongju-si, Chungcheongbuk-do (KR); CELLGENTEK CO., LTD., Cheongju-si, Chungcheongbuk-do (KR)

(72) Inventors: Suk Gil SONG, Cheongju-si, Chungcheongbuk-do (KR); Hye Ran SUNG, Cheongju-si, Chungcheongbuk-do (KR); Min Ji KIM, Sejong (KR)

(73) Assignees: CHUNGBUK NATIONAL UNIVERSITY INDUSTRY-ACADEMIC COOPERATION FOUNDATION, Cheongju-si, Chungcheongbuk-do (KR); CELLGENTEK CO., LTD., Cheongju-si, Chungcheongbuk-do (KR)

(21) Appl. No.: 17/754,122

(22) PCT Filed: Sep. 25, 2020
(86) PCT No.: PCT/KR2020/013114

§ 371 Date: Mar. 24, 2022

(30) Foreign Application Priority Data
Sep. 26, 2019 (KR) 10-2019-0118812

(51) Int. Cl. C07K 14/715; A61K 35/17; A61P 35/00

(52) U.S. Cl. CPC C07K 14/7155; C07K 14/7151; A61K 35/17; A61P 35/00; C07K 2319/03; C07K 2319/02

(57) ABSTRACT

Provided are a chimeric cytokine receptor capable of converting immunosuppressive signals to immunostimulatory signals, immune cells expressing same, and a method for treatment of cancer using the same, wherein the immune cells expressing the chimeric cytokine receptor convert immunosuppressive signals to immunostimulatory signals in a tumor microenvironment where immunosuppressive cytokines exist, thereby effectuating more potent cytotoxicity against cancer, and therefore the immune cells expressing the chimeric cytokine receptor are useful as a cell therapy product for treatment of cancer.

### TECHNICAL FIELD

The present invention relates to a chimeric cytokine receptor and a use thereof. More specifically, the present invention relates to a chimeric cytokine receptor capable of converting an immunosuppressive signal into an immune activation signal, an immune cell expressing the same, and an anticancer use thereof.

### BACKGROUND ART

Recently, studies on the anticancer effect of immune cells have been actively conducted. Natural Killer (NK) cells are effector cells specialized in the innate immune system and play an important role in a defense against cancer cells and viral infections. Natural cytotoxicity possessed by NK cells responds rapidly by appropriate stimulation of a cell membrane and is regulated by complex signaling of activating or inhibitory receptors. NK cells exhibit cytotoxicity through perforin and granzyme in a manner similar to cytotoxic T cells.

Various cytokines have been reported to have an effect on activating the differentiation, proliferation, survival, and function of NK cells, and according to study results, it has been reported that cytokines (e.g., IL-2, IL-12, IL-15, IL-18, IL-21, etc.) increase the function and activity of NK cells. Interleukin-2 (IL-2) directly acts on NK cells in a resting state, and thereby affects the proliferation of NK cells, increases cytotoxic ability, and increases the expression of perforin and IFN-γ. Interleukin-(IL-15) increases the cytotoxic ability of NK cells, affects the process of differentiation from T/NK progenitor cells into NK cells, and plays an important role in the survival and proliferation of NK cells.

Interleukin-21 (IL-21) is a cytokine secreted by activated CD4⁺ T cells, and IL-21 receptors (IL-21R, IL-21 receptors) are expressed in lymphocytes such as dendritic cells, NK, T, and B cells. IL-21 is structurally very similar to IL-2 and IL-15, and IL-21R shares a γ-chain with IL-2R, IL-15R, IL-7R, or IL-4R. IL-21 has been reported to induce the maturation of NK cell precursors from bone marrow, in particular, has been reported to increase the effect functions, such as a cytokine producing ability and cell killing ability of NK cells, and has been reported to increase the effect function of CD8⁺ T-cells, thereby promoting the anticancer response of the intrinsic and adaptive immune systems. In addition, IL-21 has been reported to activate NK cells isolated from human peripheral blood and plays an important role in inducing mature NK cells from hematopoietic stem cells isolated from umbilical cord blood.

As immunosuppressive cytokines, IL-4, IL-6, IL-10, TGF-β, etc. exist. IL-4 is a member of the γc family of cytokines, which are well known for their pro-Th2 effect during T cell differentiation. The absence of IL-4 does not affect NK cell production and homeostasis. However, NK cells express the IL-4 receptors in vitro. What should be noted is the ability of IL-4 to inhibit key NK effector functions such as cytokine production or cytotoxicity. In fact, it has been demonstrated that IL-4 inhibits the increase of production of inflammatory cytokines (IFN-γ, TNFα, and GM-CSF) induced after IL-12 treatment in human NK cells. Another measurable effect of IL-4 is to downregulate the expression of NKG2D and other NK cell activity markers in vitro and in vivo, thereby eventually reducing NKG2D-dependent cell killing. IL-4 also has an important effect on cancer development. It was found that IL-4R was significantly increased in breast cancer, prostate cancer, lung cancer, and kidney cancer, and it was also found that IL-4R was overexpressed in many types of cancer. IL-4 is an immunosuppressive cytokine, and in the case of pancreatic cancer, IL-4 uses an immune evasion strategy to produce an inhibitory cytokine, thereby limiting the persistence and function of NK and CAR-T cells.

Originally, IL-10 was known to inhibit the synthesis of cytokines in Th1 cells by being expressed and secreted in Th2 cells (J. Exp. Med. 170, 2081-2095), but now IL-10 is being reported to be produced in several types of CD4+ and CD8⁺ T cells as well as macrophages and dendritic cells (DC), B cells. The action of IL-10 is not only to mainly prevent the expression of MHCII and B7-1/2, which are required for monocytes or macrophages to present antigens or stimulate T cells, but also to inhibit the production of pro-inflammatory cytokines (e.g., IL-1α/β, IL-6, IL-12, IL-18, TNF-α, etc.) and pro-inflammatory chemokines (e.g., MCP1, MCPS, RANTES, IL-8, etc.), thereby ultimately preventing the functions of T cells and NK cells.

The patent documents and references mentioned in this specification are incorporated herein by reference to the same extent as if each document was individually and explicitly specified by reference.

### PRIOR ART DOCUMENT

(Patent Document 1) WO 2017/029512
(Non-Patent Document 1) 1. Vosshenrich C A J, Ranson T, Samson S I, Corcuff E, Colucci F, Rosmaraki E E, et al. Roles for common cytokine receptor gamma-chain-dependent cytokines in the generation, differentiation, and maturation of NK cell precursors and peripheral NK cells in vivo. J Immunol (2005) 174:1213-21.
(Non-Patent Document 2) 2. Marcenaro E, Della Chiesa M, Bellora F, Parolini S, Millo R, Moretta L, et al. IL-12 or IL-4 prime human NK cells to mediate functionally divergent interactions with dendritic cells or tumors. J Immunol (2005) 1950(174):3992-8.
(Non-Patent Document 3) 3. Brady J, Carotta S, Thong R P L, Chan C J, HayakawaY, Smyth M J, et al. The inter*?*actions of multiple cytokines control NK cell maturation. J Immunol (2010) 185:6679-88. doi:10.4049/J Immunol.0903354.
(Non-Patent Document 4) 4. Kawakami K, Kawakami M, Puri R K (2001) Overexpressed cell surface interleukin-4 receptor molecules can be successfully targeted for antitumor cytotoxin therapy. Crit Rev Immunol 21: 299-310.
(Non-Patent Document 5) 5. Gooch J L, Christy B, Yee D (2002) STAT6 mediates interleukin-4 growth inhibition in human breast cancer cells. Neoplasia 4: 324-331.

### DISCLOSURE OF THE INVENTION

### Technical Problem

The present inventors have studied and made extensive efforts to develop a chimeric cytokine receptor capable of converting an immunosuppressive signal into an immune activation signal in immune cells using gene recombination technology. As a result, they have prepared an inverted cytokine receptor (ICR), which has an extracellular domain of a cytokine receptor capable of reacting with an immunosuppressive cytokine and a transmembrane (TM)-cytoplasmic domain of an immunoactivating cytokine receptor, have successfully expressed the inverted cytokine receptor in natural killer (NK) cells, and have experimentally demonstrated the immune signal response and excellent immune activity thereof, thereby completing the present invention.

Accordingly, an object of the present invention is to provide a chimeric cytokine receptor capable of converting an immunosuppressive signal into an immune activation signal.

Another object of the present invention is to provide a polynucleotide encoding the chimeric cytokine receptor.

Still another object of the present invention is to provide a recombinant vector including the polynucleotide.

Still another object of the present invention is to provide a transformed cell expressing the chimeric cytokine receptor.

Still another object of the present invention is to provide a pharmaceutical composition for the treatment of cancer including the transformed cell as an active ingredient.

### Technical Solution

In order to solve the above objects,

the present invention provides a chimeric cytokine receptor, which includes: (i) a cytokine binding domain; (ii) a transmembrane domain; and (iii) a cytoplasmic domain; where the cytokine binding domain specifically binds to IL-4, IL-6, IL-10, or TGF-β.

Additionally, the present invention provides a chimeric cytokine receptor, which includes: (i) a cytokine binding domain; (ii) a transmembrane domain; and (iii) a cytoplasmic domain; where the cytoplasmic domain includes a cytoplasmic domain of IL-7, IL-12, IL-2/15, IL-18, or IL-21 receptor.

Additionally, the present invention provides a polynucleotide encoding the chimeric cytokine receptor.

Additionally, the present invention provides a recombinant vector including the polynucleotide.

Additionally, the present invention provides a transformed cell expressing a chimeric cytokine receptor.

Additionally, the present invention provides a pharmaceutical composition for treating or preventing cancer including the transformed cell as an active ingredient.

Hereinafter, the present invention will be described in more detail.

According to an aspect of the present invention, a chimeric cytokine receptor is provided, in which the chimeric cytokine receptor includes: (i) a cytokine binding domain; (ii) a transmembrane domain; and (iii) a cytoplasmic domain; where the cytokine binding domain specifically binds to IL-4, IL-6, IL-10, or TGF-β.

As used herein, the term “chimeric cytokine receptor” refers to a receptor in which (i) a cytokine binding domain; (ii) a transmembrane domain; And (iii) a cytoplasmic domain, which are derived from mutually-different origins, are combined and linked.

As used herein, the term “cytokine binding domain” refers to a site that specifically binds to a specific cytokine outside of a cell, and may also be referred to as the term “ectodomain” in the present specification.

As used herein, the cytokine binding domain specifically binds to IL-4, IL-6, IL-10, or TGF-β.

As used herein, the cytokine binding domain may be used without limitation as long as it is a material site capable of specifically binding to IL-4, IL-6, IL-10, or TGF-β.

According to an embodiment of the present invention, the cytokine binding domain includes an extracellular region of one or more receptors selected from the group consisting of IL-4, IL-6, IL-10, and TGF-β receptors.

According to an embodiment of the present invention, the extracellular domain of the IL-4 receptor includes the amino acid sequence of SEQ ID NO: 2.

According to an embodiment of the present invention, the extracellular domain of the IL-6 receptor includes the amino acid sequence of SEQ ID NO: 6.

According to one embodiment of the present invention, the extracellular domain of the IL-10 receptor includes the amino acid sequence of SEQ ID NO: 9.

According to an embodiment of the present invention, the extracellular domain of the TGF-β receptor includes the amino acid sequence of SEQ ID NO: 12.

As used herein, the term “transmembrane domain” refers to a site that penetrates the cell membrane and connects the cytokine binding domain and the cytoplasmic domain.

As used herein, as the transmembrane domain, any material site, which can penetrate the cell membrane and connect the cytokine domain and the cytoplasmic domain, and transmit a signal by cytokine binding to the cytoplasmic domain, may be used without limitation.

According to an embodiment of the present invention, the transmembrane domain includes a transmembrane domain of one or more receptors selected from the group consisting of IL-7, IL-12, IL-2/15, IL-18, and IL-21 receptors.

According to an embodiment of the present invention, the transmembrane domain of the IL-7 receptor includes the amino acid sequence of SEQ ID NO: 14.

According to one embodiment of the present invention, the transmembrane domain of the IL-12 receptor includes the amino acid sequence of SEQ ID NO: 16.

According to an embodiment of the present invention, the transmembrane domain of the IL-2/15 receptor includes the amino acid sequence of SEQ ID NO: 18.

According to an embodiment of the present invention, the transmembrane domain of the IL-18 receptor includes the amino acid sequence of SEQ ID NO: 20.

According to an embodiment of the present invention, the transmembrane domain of the IL-21 receptor includes the amino acid sequence of SEQ ID NO: 22.

As used herein, the term “cytoplasmic domain (endodomain)” refers to a site which, being located inside a cell, serves the function of converting a cytokine binding signal transmitted through the transmembrane domain into a signal located inside a cell.

As used herein, the cytoplasmic domain may be used without limitation, as long as it is a site that serves the function of converting and transmitting a signal by cytokine binding into a cell.

According to an embodiment of the present invention, the cytoplasmic domain includes a cytoplasmic domain of one or more receptors selected from the group consisting of IL-7, IL-12, IL-2/15, IL-18, and IL-21 receptors.

According to an embodiment of the present invention, the cytoplasmic domain of the IL-7 receptor includes the amino acid sequence of SEQ ID NO: 15.

According to an embodiment of the present invention, the cytoplasmic domain of the IL-12 receptor includes the amino acid sequence of SEQ ID NO: 17.

According to an embodiment of the present invention, the cytoplasmic domain of the IL-2/15 receptor includes the amino acid sequence of SEQ ID NO: 19.

According to an embodiment of the present invention, the cytoplasmic domain of the IL-18 receptor includes the amino acid sequence of SEQ ID NO: 21.

According to one embodiment of the present invention, the intracellular domain of the IL-21 receptor includes the amino acid sequence of SEQ ID NO: 23.

The chimeric cytokine receptor of the present invention may further include a signal peptide.

As used herein, the term “signal peptide” refers to a peptide that functions to move and localize the expressed chimeric cytokine receptor to the cell membrane domain, and another term known in the art “signal sequence (signal sequence)”, “targeting signal”, and “localization signal”.

According to an embodiment of the present invention, the signal peptide is linked to the cytokine binding domain of a chimeric cytokine receptor, and is preferably linked to the N-terminus of the cytokine binding domain.

According to an embodiment of the present invention, the signal peptide may include a signal peptide of one or more receptors selected from the group consisting of IL-4, IL-6, IL-10, and TGF-β receptors.

According to an embodiment of the present invention, the signal peptide of the IL-4 receptor may include the amino acid sequence of SEQ ID NO: 1.

According to an embodiment of the present invention, the signal peptide of the IL-6 receptor may include the amino acid sequence of SEQ ID NO: 5.

According to an embodiment of the present invention, the signal peptide of the IL-10 receptor may include the amino acid sequence of SEQ ID NO: 8.

According to an embodiment of the present invention, the signal peptide of the TGF-β receptor may include the amino acid sequence of SEQ ID NO: 11.

In an embodiment of the present invention, the chimeric cytokine receptor of the present invention can convert a signal of an immunosuppressive cytokine into an immunoactivating signal, and in this sense, the chimeric cytokine receptor may be referred to as another term “an inverted chimeric receptor”.

According to another aspect of the present invention, a polynucleotide encoding the chimeric cytokine receptor of the present invention is provided.

As used herein, the term “coding” means a polynucleotide referred to as “coding for a polypeptide” when it can be transcribed and/or translated to produce mRNA for the polypeptide and/or a fragment thereof where it is manipulated by a method well known to those skilled in the art or where it is naturally occurred.

As used herein, the term “polynucleotide” is used interchangeably and refers to a polymer form of nucleotides of any length among ribonucleotides or deoxyribonucleotides. The term polynucleotide refers to single, double, or multi-stranded DNA or RNA, genomic DNA, cDNA, a DNA-RNA hybrid, or a polymer including purine and pyrimidine bases or other natural, chemically or biochemically modified, unnatural, or derivatized nucleotide base, but is not limited thereto.

It will be well understood by those skilled in the art that the polynucleotide encoding the chimeric cytokine receptor of the present invention can undergo various modifications in the coding region within the range not altering the amino acid sequence of the chimeric cytokine receptor expressed from the coding region, due to codon degeneracy or in consideration of the preferred codons in the organism to express the chimeric cytokine receptor, and various modifications can be made within the range not affecting the expression of the gene even in parts excluding the coding domain, and that such modified genes are also included in the scope of the present invention. That is, as long as the polynucleotide of the present invention encodes a protein having equivalent activity, one or more nucleic acid bases may be modified by substitution, deletion, insertion, or a combination thereof, and these are also included in the scope of the present invention.

According to still another aspect of the present invention, a recombinant vector, which includes a polynucleotide encoding a chimeric cytokine receptor of the present invention, is provided.

As the vector used in the present invention, various vectors known in the art can be used. Additionally, according to the type of host cell to produce the chimeric cytokine receptor promoter, expression control sequences (e.g., terminator, enhancer, etc.), sequences for membrane targeting or secretion, etc. may be appropriately selected and variously combined according to the purpose.

In the present invention, the vector includes a plasmid vector, a cosmid vector, a bacteriophage vector, a viral vector, etc., but is not limited thereto. A suitable recombinant vector may include a signal sequence or leader sequence for membrane targeting or secretion in addition to expression control elements (e.g., promoter, operator, start codon, stop codon, polyadenylation signal, and enhancer), and can be prepared in various ways according to the purpose.

In the present invention, the vector includes as a selection marker an antibiotic resistance gene commonly used in the art (e.g., genes resistant to ampicillin, gentamicin, carbenicillin, chloramphenicol, streptomycin, kanamycin, geneticin, neomycin, puromycin, and tetracycline).

According to still another aspect of the present invention, a transformed cell expressing the chimeric cytokine receptor of the present invention is provided.

According to an embodiment of the present invention, the transformed cell expressing the chimeric cytokine receptor may be a cell transformed with the recombinant vector of the present invention.

In the present invention, as a method of introducing a recombinant vector into a cell, a known transfection method may be used, which includes, for example, a microinjection method (Capecchi, MR, Cell 22, 479 (1980)), a calcium phosphate precipitation method (Graham, F L et al., Virology 52, 456 (1973)), an electroporation method (Neumann, E. et al., EMBO J. 1, 841 (1982)), liposome-mediated transfection (Wong, T K et al. Gene, 10, 87 (1980)), a DEAE-dextran treatment method (Gopal, Mol. Cell Biol. 5, 1188-1190 (1985)), Gene Bombardment (Yang et al., Proc. Natl. Acad. Sci. USA 87, 9568-9572 (1990)), etc., but are not limited thereto.

In the present invention, the cell into which the recombinant vector can be introduced may be an immune cell, more preferably a natural killer (NK) cell, a T cell, a cytotoxic T cell, a regulatory T cell, or a B cell, or an NK-T cell, more preferably an NK cell or T cell. Preferably, the cell may be a human-derived immune cell, more preferably a human-derived NK cell.

As used herein, the term “T cell” refers to a type of lymphocyte that matures in the thymus. T cells play an important role in cell-mediated immunity and are distinguished from other lymphocytes (e.g., B lymphocytes) by the presence of T cell receptors on the cell surface. T cells can also be isolated or obtained from commercially available sources. T cells include all types of CD3 expressing CD3 including helper T cells (CD4⁺ cells), cytotoxic T cells (CD8⁺ cells), natural killer T cells, regulatory T cells (Treg), and gamma-delta T cells. “Cytotoxic cells” include CD8⁺ T cells, natural-killer (NK) cells, and neutrophils that can mediate cytotoxic responses.

As used herein, the term “NK cell” is also known as a natural killer cell, and refers to a type of lymphocyte derived from the bone marrow, which plays an important role in the innate immune system. Even in the absence of a major histocompatibility complex or antibody on the cell surface, NK cells provide a rapid immune response to virus-infected cells, tumor cells, or other stressed cells. Non-limiting examples of commercial NK cell lines include NK-92 (ATCC® CRL-2407™) and NK-92MI (ATCC® CRL-2408™). Additional examples include the NK cell lines (e.g., HANK1, KHYG-1, NKL, NK-YS, NOI-90, YT, and NK101), but are not limited thereto. Non-limiting exemplary sources of such commercially available cell lines include the American Type Culture Collection, or ATCC, (http://www.atcc.org/) and the German Collection of Microorganisms and Cell Cultures (https://www.dsmz.de/).

In the present invention, the step of selecting the transformed cells can easily be performed using a phenotype expressed by the above-described vector selection label. For example, when the selection marker is a specific antibiotic resistance gene, transformed cells can easily be selected by culturing a transformant in a medium containing the antibiotic.

According to still another aspect of the present invention, there is provided a pharmaceutical composition for the treatment or prevention of cancer, which includes cells expressing the above-described chimeric cytokine receptor as an active ingredient.

As used herein, the term “treatment” means (a) inhibition of the development of a disorder or disease; (b) alleviation of a disorder or disease; and (c) elimination of a disorder or disease.

As used herein, the term “prevention” means inhibiting the occurrence of a disorder or disease in an animal, which has not been diagnosed as possessing a disorder or disease but is prone to such a disorder or disease.

According to an embodiment of the present invention, the cancer may be, as non-limiting examples, a cancer selected from the group consisting of breast cancer, lung cancer, gastric cancer, liver cancer, gallbladder cancer, blood cancer, Hodgkin's and non-Hodgkin's lymphoma, chronic lymphocytic leukemia, acute lymphoblastic leukemia, chronic myeloid leukemia, acute myeloblastic leukemia, bone cancer, pancreatic cancer, skin cancer, head and neck cancer, ocular melanoma, uterine sarcoma, rectal cancer, anal cancer, colorectal cancer, fallopian tube cancer, endometrial cancer, cervical cancer, ovarian cancer, small intestine cancer, endocrine cancer, thyroid cancer, parathyroid cancer, kidney cancer, soft tissue tumor, urethral cancer, prostate cancer, bronchial cancer, bone marrow cancer, and multiple myeloma.

The pharmaceutical composition of the present invention may be prepared as an injection, typically in the form of a suspension including cells. Pharmaceutical forms suitable for injection include sterile aqueous solutions or dispersions ready for immediate preparation of solutions or dispersions. In all cases, pharmaceuticals in the form of injection solutions must be sterile and must have flowability to facilitate injection.

The pharmaceutical composition of the present invention may include a pharmaceutically acceptable carrier in addition to an active ingredient.

As used herein, the term “pharmaceutically acceptable” means that it does not cause an allergic reaction or similar adverse reaction when administered to humans. Such carriers include specific solvents, dispersion media, coating agents, antibacterial and antifungal agents, isotonic and absorption delaying agents, etc. It is known in the art to use such media and agents for pharmaceutically active materials.

The carrier of the pharmaceutical composition may be, for example, water, saline, ethanol, polyol (e.g., glycerol, propylene glycol, liquid polyethylene glycol, etc.), a suitable mixture thereof, and a solvent or dispersion medium including vegetable oil. Flowability can be maintained by the use of a coating agent (e.g., lecithin). In order to prevent microbial contamination, various antibacterial and antifungal agents (e.g., paraben, chlorobutanol, phenol, sorbic acid, thimerosal, etc.) may be included, and an isotonic agent (e.g., sugar, sodium chloride, etc.) may also be included. In addition, agents that delay absorption (e.g., aluminum monostearate and gelatin) may be included in the composition so as to prolong the absorption effect upon administration to the body. Sterile injection solutions are prepared by mixing a required amount of the active compound in a suitable solvent having the various other ingredients mentioned above as necessary, followed by sterilization and filtration.

The pharmaceutical composition of the present invention may preferably be administered by parenteral, intraperitoneal, intradermal, intramuscular, or intravenous route.

The pharmaceutical composition of the present invention is administered in a therapeutically effective amount in a manner compatible with the formulation. In addition, the dose may be adjusted according to the state or condition of the subject to be treated. For parenteral administration as an aqueous injection solution, the solution must be suitably buffered as needed, and the liquid diluent is first made isotonic with sufficient saline or glucose. These special aqueous solutions are particularly suitable for intravenous, intramuscular, subcutaneous, intradermal, and intraperitoneal administration.

Information on carriers, agents, and media that can be used in the pharmaceutical composition of the present invention is known in the art (see “Remington's Pharmaceutical Sciences”, 1995, 15th edition).

### Advantageous Effects

The features and advantages of the present invention are summarized as follows:

(i) The present invention relates to a chimeric cytokine receptor capable of converting an immunosuppressive signal into an immune activation signal, an immune cell expressing the same, and a pharmaceutical composition for the treatment of cancer including the immune cell as an active ingredient.

(ii) Immune cells expressing the chimeric cytokine receptor of the present invention can exhibit a stronger cytotoxic effect against cancer by converting an immunosuppressive signal into an immune activation signal in a microtumor environment in which an immunosuppressive cytokine is present.

(iii) The immune cells expressing the chimeric cytokine receptor of the present invention can be used as a cell therapy agent for cancer treatment.

### BRIEF DESCRIPTION OF THE DRAWINGS

FIGS. 1A and 1B each show the results of confirming the expression of each receptor in NK cells prepared to express the chimeric cytokine receptor in the present invention at the RNA level by RT-PCR (FIG. 1A), and the results of confirming the receptor expressed in the cell membrane of NK cells by flow cytometry (FIG. 1B).

FIG. 2 shows the results of the amount of interferon-gamma (IFN-γ), produced in NK cells after treatment with IL-4 (i.e., an immunosuppressive cytokine) measured by enzyme-linked immunosorbent assay (ELISA) so as to confirm the immune activity of chimeric cytokine receptor-expressing NK cells in an environment in which an immunosuppressive signal exists (e.g., a microtumor environment).

FIGS. 3A and 3B each show the results of confirming the changes in the expression of immunoactivating markers, such as DNAM-1 and NKp46, in NK cells expressing chimeric cytokine receptors by IL-4 (i.e., an immunosuppressive cytokine).

FIGS. 4A, 4B, and 4C each show the results of confirming the cytotoxicity of NK cells expressing chimeric cytokine receptors by confirming the change in granzyme B (FIG. 4C) and interferon-gamma (IFN-γ) (FIG. 4B) produced by NK cells, and cancer cell killing ability (FIG. 4A) by reacting NK cells expressing chimeric cytokine receptors with K562 cancer cells.

### MODE FOR CARRYING OUT THE INVENTION

The specific embodiments described herein are meant to represent preferred embodiments or examples of the present invention, and the scope of the present invention is not limited thereto. It will be apparent to those skilled in the art that variations and other uses of the present invention do not depart from the scope of the inventions described in the claims of this specification.

### EXAMPLES

### Example 1. Preparation of NK Cells Expressing Inverted Cytokine Receptor (ICR)

The amino acid sequences of cytokine receptors that can be used to constitute chimeric cytokine receptors or inverted cytokine receptors (ICRs) in the present invention are shown in Table 1 below.

In order to prepare NK cells expressing chimeric cytokine receptors, which are capable of transmitting an immune activation signal in NK cells by binding with IL-4 (i.e., an immunosuppressive cytokine) in a microtumor environment, an inverted cytokine receptor (ICR) was designed by a combination of peptides constituting cytokine receptors, and ICR_1, ICR_2, and ICR_3, which encode this ICR, were synthesized. The structure of each gene is as follows, and each amino acid sequence is shown in Table 1 below.

ICR_1 consists of a signal peptide of an IL-4 receptor (SEQ ID NO: 1), an extracellular domain an IL-4 receptor (SEQ ID NO: 2), a transmembrane domain of an IL-7 receptor (SEQ ID NO: 14), and a cytoplasmic domain an IL-7 receptor (SEQ ID NO: 15).

ICR_2, which is a control group of this example, consists of a signal peptide of an IL-4 receptor (SEQ ID NO: 1), an extracellular domain of an IL-4 receptor (SEQ ID NO: 2), and a transmembrane domain of an IL-4 receptor (SEQ ID NO: 3), in which the cytoplasmic domain of the IL-4 receptor is deleted.

ICR_3 consists of a signal peptide of an IL-4 receptor (SEQ ID NO: 1), an extracellular domain of an IL-4 receptor (SEQ ID NO: 2), a transmembrane domain of an IL-21 receptor (SEQ ID NO: 22), and a cytoplasmic domain of an IL-21 receptor (SEQ ID NO: 23).

Each amino acid sequence consisting of a combination of several cytokine receptors was synthesized by converting into a nucleotide sequence having a codon sequence optimized for humans, and cloned into a vector to thereby obtain each inverted cytokine receptor (ICR) gene.

In order to prepare NK cells which express chimeric cytokine receptors (that bind to immunosuppressive cytokines and transmit immune activation signals into immune cells) or inverted cytokine receptors (ICRs), each inverted cytokine receptor (ICR) gene was transfected into NK cells according to the manufacturer's protocol using the LONZA's Nucleofector Cell Line Nucleofector® Kit. After the transformation, the cells were stabilized for 48 hours in the MEM alpha medium containing 0.1 mM 2-mercaptoethanol and 100 U/mL of recombinant interleukin-2 (IL-2), and then cell lines were established by conducting selection culture of only the transformed cells using antibiotics such as puromycin or geneticin (G418).

TABLE 1

| SEQ | | |
|---|---|---|
| ID | Sequence | |
| NO | Information | Description |
| 1 | MGWLCSGLLFPVSCLVLLQV | IL-4R |
| | ASSGN | signal |
| | | peptide |
| 2 | MKVLQEPTCVSDYMSISTCE | IL-4R |
| | WKMNGPTNCSTELRLLYQLV | extracellular |
| | FLLSEAHTCIPENNGGAGCV | domain |
| | CHLLMDDVVSADNYTLDLWA | |
| | GQQLLWKGSFKPSEHVKPRA | |
| | PGNLTVHTNVSDTLLLTWSN | |
| | PYPPDNYLYNHLTYAVNIWS | |
| | ENDPADFRIYNVTYLEPSLR | |
| | IAASTLKSGISYRARVRAWA | |
| | QCYNTTWSEWSPSTKWHNSY | |
| | REPFEQH | |
| 3 | LLLGVSVSCIVILAVCLLCY | IL-4R |
| | VSIT | transmembrane |
| | | domain |
| 4 | KIKKEWWDQIPNPARSRLVA | IL-4R |
| | IIIQDAQGSQWEKRSRGQEP | cytoplasmic |
| | AKCPHWKNCLTKLLPCFLEH | domain |
| | NMKRDEDPHKAAKEMPFQGS | |
| | GKSAWCPVEISKTVLWPESI | |
| | SVVRCVELFEAPVECEEEEE | |
| | VEEEKGSFCASPESSRDDFQ | |
| | EGREGIVARLTESLFLDLLG | |
| | EENGGFCQQDMGESCLLPPS | |
| | GSTSAHMPWDEFPSAGPKEA | |
| | PPWGKEQPLHLEPSPPASPT | |
| | QSPDNLTCTETPLVIAGNPA | |
| | YRSFSNSLSQSPCPRELGPD | |
| | PLLARHLEEVEPEMPCVPQL | |
| | SEPTTVPQPEPETWEQILRR | |
| | NVLQHGAAAAPVSAPTSGYQ | |
| | EFVHAVEQGGTQASAVVGLG | |
| | PPGEAGYKAFSSLLASSAVS | |
| | PEKCGFGASSGEEGYKPFQD | |
| | LIPGCPGDPAPVPVPLFTFG | |
| | LDREPPRSPQSSHLPSSSPE | |
| | HLGLEPGEKVEDMPKPPLPQ | |
| | EQATDPLVDSLGSGIVYSAL | |
| | TCHLCGHLKQCHGQEDGGQT | |
| | PVMASPCCGCCCGDRSSPPT | |
| | TPLRAPDPSPGGVPLEASLC | |
| | PASLAPSGISEKSKSSSSFH | |
| | PAPGNAQSSSQTPKIVNFVS | |
| | VGPTYMRVS | |
| 5 | MLAVGCALLAALLAAPGAA | IL-6R |
| | | signal |
| | | peptide |
| 6 | LAPRRCPAQEVARGVLTSLP | IL-6R |
| | GDSVTLTCPGVEPEDNATVH | extracellular |
| | WVLRKPAAGSHPSRWAGMGR | domain |
| | RLLLRSVQLHDSGNYSCYRA | |
| | GRPAGTVHLLVDVPPEEPQL | |
| | SCFRKSPLSNVVCEWGPRST | |
| | PSLTTKAVLLVRKFQNSPAE | |
| | DFQEPCQYSQESQKFSCQLA | |
| | VPEGDSSFYIVSMCVASSVG | |
| | SKFSKTQTFQGCGILQPDPP | |
| | ANITVTAVARNPRWLSVTWQ | |
| | DPHSWNSSFYRLRFELRYRA | |
| | ERSKTFTTWMVKDLQHHCVI | |
| | HDAWSGLRHVVQLRAQEEFG | |
| | QGEWSEWSPEAMGTPWTESR | |
| | SPPAENEVSTPMQALTTNKD | |
| | DDNILFRDSANATSLPVQDS | |
| | SSVPLP | |
| 7 | TFLVAGGSLAFGTLLCIAIV | IL-6R |
| | L | transmembrane |
| | | domain |
| 8 | MLPCLWLLAALLSLRLGSDA | IL-10R |
| | | signal |
| | | peptide |
| 9 | GSDAHGTELPSPPSVWFEAE | IL-10R |
| | FFHHILHWTPIPNQSESTCY | extracellular |
| | EVALLRYGIESWNSISNCSQ | domain |
| | TLSYDLTAVTLDLYHSNGYR | |
| | ARVRAVDGSRHSNWTVTNTR | |
| | FSVDEVTLTVGSVNLEIHNG | |
| | FILGKIQLPRPKMAPANDTY | |
| | ESIFSHFREYEIAIRKVPGN | |
| | FTFTHKKVKHENFSLLTSGE | |
| | VGEFCVQVKPSVASRSNKGM | |
| | WSKEECISLTRQYFTVTN | |
| 10 | VIIFFAFVLLLSGALAYCLA | IL-10R |
| | L | transmembrane |
| | | domain |
| 11 | MGRGLLRGLWPLHIVLWTRI | TGF-βR II |
| | AS | signal |
| | | peptide |
| 12 | TIPPHVQKSVNNDMIVTDNN | TGF-βR II |
| | GAVKFPQLCKFCDVRFSTCD | extracellular |
| | NQKSCMSNCSITSICEKPQE | domain |
| | VCVAVWRKNDENITLETVCH | |
| | DPKLPYHDFILEDAASPKCI | |
| | MKEKKKPGETFFMCSCSSDE | |
| | CNDNIIFSEEYNTSNPDLLL | |
| | VIFQ | |
| 13 | VTGISLLPPLGVAISVIIIF | TGF-βR II |
| | Y | transmembrane |
| | | domain |
| 14 | PILLTISILSFFSVALLVIL | IL-7R |
| | ACVLW | transmembrane |
| | | domain |
| 15 | KKRIKPIVWPSLPDHKKTLE | IL-7R |
| | HLCKKPRKNLNVSFNPESFL | cytoplasmic |
| | DCQIHRVDDIQARDEVEGFL | domain |
| | QDTFPQQLEESEKQRLGGDV | |
| | QSPNCPSEDVVITPESFGRD | |
| | SSLTCLAGNVSACDAPILSS | |
| | SRSLDCRESGKNGPHVYQDL | |
| | LLSLGTTNSTLPPPFSLQSG | |
| | ILTLNPVAQGQPILTSLGSN | |
| | QEEAYVTMSSFYQNQ | |

### Example 2. Confirmation of Expression of Chimeric Receptor in NK Cells Introduced with Inverted Cytokine Receptor (ICR)

In order to confirm the expression of the inverted cytokine receptors (ICRs) in the NK cells prepared in this experiment into which ICRs were introduced, the mRNA expression and flow cytometry were performed as follows.

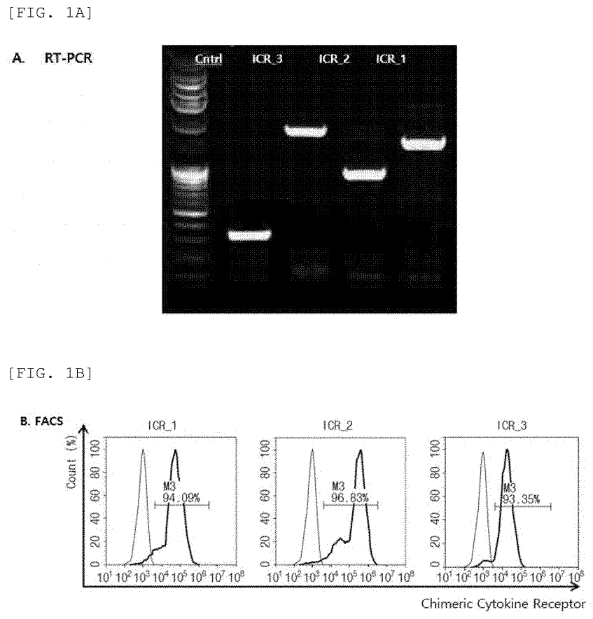

After extracting mRNA from NK cells to synthesize cDNA, the expression of inverted cytokine receptors (ICRs) in each NK cell was confirmed by RT-PCR using a primer set, that is, the forward primer 5′-GCCTCAGACAGTGGTTCAAAC-3′ (SEQ ID NO: 24) and the reverse primer 5′-AGGCACAGTCGAGGCTGAT-3′ (SEQ ID NO: 25) (FIG. 1A). The expression of each inverted cytokine receptor (ICR) was confirmed by flow cytometry (NovoCyte Flow Cytometer, ACEA Biosciences Inc.) using an antibody that binds to the extracellular domain of the IL-4 receptor, which is a site where the IL-4 binds to cytokines, commonly possessed by the ICR-expressing NK cells (ICR_1, ICR_2, and ICR_3) (FIG. 1B).

### Example 3. Confirmation of Changes in Characteristics of NK Cells Expressing Inverted Cytokine Receptor (ICR) by Immunosuppressive Cytokines

In order to compare the function and activity of NK cells and ICR-NK cells in an environment similar to a tumor microenvironment in which a large amount of immunosuppressive cytokines exist, the changes in characteristics and activity of NK cells were confirmed NK cells by artificially treating them with the immunosuppressive cytokine IL-4.

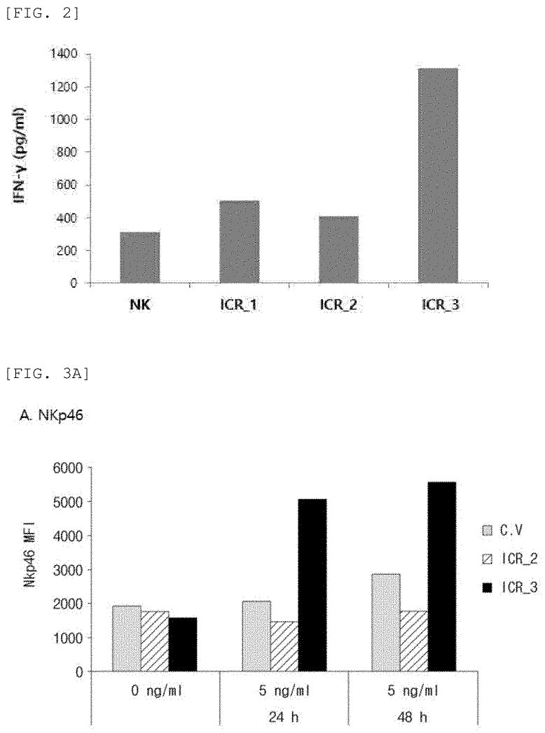

First, in order to confirm the immune activity, by immunosuppressive cytokines, of the three types of NK cells (ICR_1, ICR_2, and ICR_3), in which the expression of the inverted cytokine receptor (ICR) was confirmed, each NK cell was treated with 5 ng/mL of IL-4 for 24 hours and the amount of interferon-gamma (IFN-γ) was measured to compare the immune activity of each NK cell. As a result, it was confirmed that the amount of interferon-gamma (IFN-γ) production was significantly increased in ICR_3 NK cells expressing chimeric receptors having a cytoplasmic domain of the IL-21 receptor, compared to other NK cells (FIG. 2).

Among the receptors of NK cells, there are activating receptors (which transmit signals that activate NK cells by reacting with target cells) and inhibitory receptors (which, on the contrary, transmit signals that inhibit the activity of NK cells), and the balance of these receptors regulates the immune activity of NK cells. When the expression of active receptors increases on the surface of NK cells, the immune activation signal is strongly transmitted into the cell, thereby strongly inducing the immune activity of NK cells.

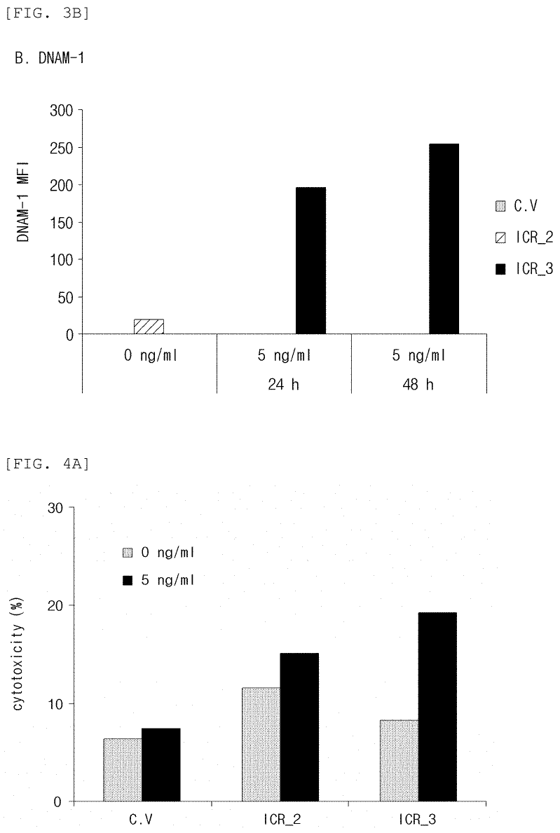

In order to compare the changes in expression of NK cell-activating receptors according to the presence/absence of IL-4 cytokine in NK cells expressing inverted cytokine receptor (ICR), the expression level of NKp30, NKp44, NKp46, and DNAM-1 on the surface of NK cells was confirmed using FACS (NovoCyte 3000, ACEA Bioscience Inc.). After incubating in an incubator at 5% CO₂ at 37° C. in NK cell culture medium with or without IL-4 cytokine treatment for 24 hours or 48 hours, the NK cells were washed twice with PBS, and each antibody was added thereto and allowed to react for 30 minutes in a state where the light is blocked, and the cells were washed twice with PBS again. NK cells were suspended in 1% BSA/PBS and analyzed using FACS. When the cells were treated with immunosuppressive cytokine IL-4 for 24 hours or 48 hours, the expression of other active receptors in ICR_3 cells was weakly increased or decreased on the contrary, but the expression of NKp46 and DNAM-1 was significantly increased (FIGS. 3A and 3B). In contrast, there was no change in the expression level of inhibitory receptors (e.g., CD158a (KIR2DL1), CD158b (KIR2DL2/DL3), and CD159a (NKG2A)) (data not shown).

### Example 4. Cytotoxicity of NK Cells Expressing Inverted Cytokine Receptor (ICR)

In this experiment, the cytotoxicity of ICR_3 NK cells was measured compared to C.V (NK cells transformed with empty vector) and ICR_2 NK cells as a control to confirm the cytotoxicity of NK cells introduced with the inverted cytokine receptor (ICR).

As a method for cytotoxicity analysis, carboxyfluorescein succinimidyl ester (CFSE) was added to K562 target cells (T) to a final concentration of 0.5 μM per 1×10⁶ cells, and the cells were stained in the environment at 5% CO₂ at 37° C. for 30 minutes, washed 3 times with PBS, and dispensed into a 96-well round bottom plate at a density of 4×10⁴ cells. The NK cells (effector cells; E), which were cultured in an incubator at 5% CO₂ at 37° C. for 48 hours with or without IL-4 cytokine treatment, were reacted with the target cells at a 1:1 E:T ratio for 4 hours, and the cytotoxicity of the NK cells was measured as follows. After washing the cells 3 times with PBS, they were suspended in 100 μL of 1% BSA/PBS, and 5 μL of 7-AAD was added to each well and reacted at 4° C. for 30 minutes in a state where the light was blocked, and then the cells were washed again twice using PBS. Thereafter, the NK cells were suspended in 1% BSA/PBS, and the cytotoxicity by NK cells was compared and analyzed using flow cytometry.

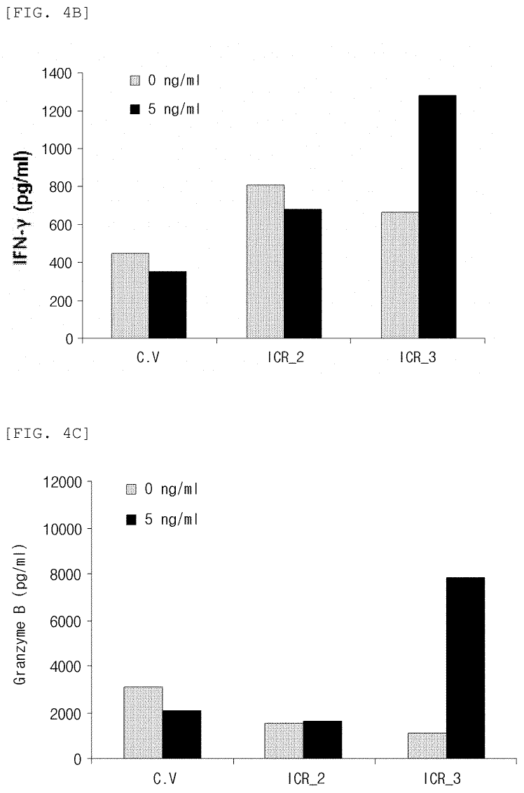

The comparison of the cytotoxicity of cells treated or untreated with IL-4 revealed that the ICR_3 NK cells treated with IL-4 for 48 hours showed an about 2.5-fold increase of cytotoxicity to K562 target cells compared to those without IL-4 (FIG. 4A). In particular, the amounts of granzyme B and interferon-gamma (IFN-γ) produced in NK cells were measured by ELISA. As a result, it was confirmed that granzyme and IFN-γ were also secreted in a specifically high amount in ICR_3 NK cells treated with IL-4 for 48 hours (FIGS. 4B and 4C).

Based on the above experimental results, it was confirmed that the NK cells expressing ICR_3 inverted cytokine receptor (ICR) prepared in the present invention could exhibit anticancer efficacy much superior to that of conventional NK cells by transmitting an immune activation signal into cells by way of a reverse use of immunosuppressive signals by immunosuppressive cytokines in a microtumor environment where immunosuppressive cytokines are enriched.

As described above, specific parts of the present invention have been described in detail, and it is apparent that these specific techniques are only preferred embodiments for those of ordinary skill in the art, and the scope of the present invention is not limited thereto. Accordingly, it should be noted that the substantial scope of the present invention is defined by the appended claims and equivalents thereof.

1. A chimeric cytokine receptor comprising:
(i) a cytokine binding domain;
(ii) a transmembrane domain; and
(iii) a cytoplasmic domain;
wherein the cytokine binding domain specifically binds to IL-4, IL-6, IL-10, or TGF-β.

2. The chimeric cytokine receptor of claim 1, wherein the cytokine binding domain comprises an extracellular domain of one or more receptors selected from the group consisting of IL-4, IL-6, IL-10, and TGF-β receptors.

3. The chimeric cytokine receptor of claim 2, wherein:
the extracellular domain of the IL-4 receptor comprises an amino acid sequence of SEQ ID NO: 2;
the extracellular domain of the IL-6 receptor comprises an amino acid sequence of SEQ ID NO: 6;
the extracellular domain of the IL-10 receptor comprises an amino acid sequence of SEQ ID NO: 9; and
the extracellular domain of the TGF-β receptor comprises an amino acid sequence of SEQ ID NO: 12.

4. The chimeric cytokine receptor of claim 1, wherein the transmembrane domain comprises a transmembrane domain of one or more receptors selected from the group consisting of IL-7, IL-12, IL-2/15, IL-18, and IL-21 receptors.

5. The chimeric cytokine receptor of claim 4, wherein:
the transmembrane domain of the IL-7 receptor comprises an amino acid sequence of SEQ ID NO: 14;
the transmembrane domain of the IL-12 receptor comprises an amino acid sequence of SEQ ID NO: 16;
the transmembrane domain of the IL-2/15 receptor comprises an amino acid sequence of SEQ ID NO: 18;
the transmembrane domain of the IL-18 receptor comprises an amino acid sequence of SEQ ID NO: 20; and
the transmembrane domain of the IL-21 receptor comprises an amino acid sequence of SEQ ID NO: 22.

6. The chimeric cytokine receptor of claim 1, wherein the cytoplasmic domain comprises a cytoplasmic domain of one or more receptors selected from the group consisting of IL-7, IL-12, IL-2/15, IL-18, and IL-21 receptors.

7. The chimeric cytokine receptor of claim 6, wherein:
the cytoplasmic domain of the IL-7 receptor comprises an amino acid sequence of SEQ ID NO: 15;
the cytoplasmic domain of the IL-12 receptor comprises an amino acid sequence of SEQ ID NO: 17;
the cytoplasmic domain of the IL-2/15 receptor comprises an amino acid sequence of SEQ ID NO: 19;
the cytoplasmic domain of the IL-18 receptor comprises an amino acid sequence of SEQ ID NO: 21; and
the cytoplasmic domain of the IL-21 receptor comprises an amino acid sequence of SEQ ID NO: 23.

8. The chimeric cytokine receptor of claim 1, wherein the cytokine binding domain further comprises a signal peptide.

9. The chimeric cytokine receptor of claim 8, wherein the signal peptide comprises a signal peptide of one or more receptors selected from the group consisting of IL-4, IL-6, IL-10, and TGF-β receptors.

10. The chimeric cytokine receptor of claim 9, wherein:
the signal peptide of the IL-4 receptor comprises an amino acid sequence of SEQ ID NO: 1;
the signal peptide of the IL-6 receptor comprises an amino acid sequence of SEQ ID NO: 5;
the signal peptide of the IL-10 receptor comprises an amino acid sequence of SEQ ID NO: 8; and
the signal peptide of the TGF-β receptor comprises an amino acid sequence of SEQ ID NO: 11.

11. A polynucleotide encoding the chimeric cytokine receptor described in claim 1.

12. A recombinant vector comprising the polynucleotide described in claim 11.

13. A transformed cell expressing the chimeric cytokine receptor described in claim 1.

14. The transformed cell of claim 13, wherein the cell is an NK cell, a T cell, a cytotoxic T cell, or a regulatory T cell.

15. A method
for treating or preventing cancer comprising administering to a subject in need thereof, a pharmaceutical composition comprising an effective amount of the cell according to claim 13 as an active ingredient.

16. The method of claim 15, wherein the cancer is a cancer selected from the group consisting of breast cancer, lung cancer, gastric cancer, liver cancer, gallbladder cancer, blood cancer, Hodgkin's and non-Hodgkin's lymphoma, chronic lymphocytic leukemia, acute lymphoblastic leukemia, chronic myeloid leukemia, acute myeloblastic leukemia, bone cancer, pancreatic cancer, skin cancer, head and neck cancer, skin cancer, ocular melanoma, uterine sarcoma, ovarian cancer, rectal cancer, anal cancer, colorectal cancer, fallopian tube cancer, endometrial cancer, cervical cancer, ovarian cancer, small intestine cancer, endocrine cancer, thyroid cancer, parathyroid cancer, kidney cancer, soft tissue tumor, urethral cancer, prostate cancer, bronchial cancer, bone marrow cancer, and multiple myeloma.


SEQUENCE LISTING (25 sequences)

SEQ ID NO: 1 - protein, 25 aa, Homo sapiens
MGWLCSGLLFPVSCLVLLQVASSGN

SEQ ID NO: 2 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 3 - protein, 24 aa, Homo sapiens
LLLGVSVSCIVILAVCLLCYVSIT

SEQ ID NO: 4 - protein, 569 aa, Homo sapiens
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 5 - protein, 19 aa, Homo sapiens
MLAVGCALLAALLAAPGAA

SEQ ID NO: 6 - protein, 346 aa, Homo sapiens
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLP

SEQ ID NO: 7 - protein, 21 aa, Homo sapiens
TFLVAGGSLAFGTLLCIAIVL

SEQ ID NO: 8 - protein, 21 aa, Homo sapiens
MLPCLVVLLAALLSLRLGSDA

SEQ ID NO: 9 - protein, 218 aa, Homo sapiens
GSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 10 - protein, 21 aa, Homo sapiens
VIIFFAFVLLLSGALAYCLAL

SEQ ID NO: 11 - protein, 22 aa, Homo sapiens
MGRGLLRGLWPLHIVLWTRIAS

SEQ ID NO: 12 - protein, 144 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 13 - protein, 21 aa, Homo sapiens
VTGISLLPPLGVAISVIIIFY

SEQ ID NO: 14 - protein, 25 aa, Homo sapiens
PILLTISILSFFSVALLVILACVLW

SEQ ID NO: 15 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 16 - protein, 21 aa, Homo sapiens
WMAFVAPSICIAIIMVGIFST

SEQ ID NO: 17 - protein, 219 aa, Homo sapiens
HYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 18 - protein, 25 aa, Homo sapiens
IPWLGHLLVGLSGAFGFIILVYLLI

SEQ ID NO: 19 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 20 - protein, 21 aa, Homo sapiens
GVVLLYILLGTIGTLVAVLAA

SEQ ID NO: 21 - protein, 222 aa, Homo sapiens
SALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 22 - protein, 21 aa, Homo sapiens
GWNPHLLLLLLLVIVFIPAFW

SEQ ID NO: 23 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 24 - DNA, 21 bp, Artificial Sequence
  PCR forward primer
gcctcagacagtggttcaaac

SEQ ID NO: 25 - DNA, 19 bp, Artificial Sequence
  PCR reverse primer
aggcacagtcgaggctgat